(19) United States
(12) Patent Application Publication
(10) Pub. No.: US 2003/0045482 A1
(43) Pub. Date: Mar. 6, 2003

(54) Using D-ribose with or without anti-microbial agents to enhance healing and subsequent recovery by both synthesizing and sparing NAD derivatives

(76) Inventor: Keith E. Kenyon, Van Nuys, CA (US)

Correspondence Address: Keith E. Kenyon, M.D., Suite C, 14435 Hamlin Street, Van Nuys, CA, 91401-6205, US

(21) Appl. No.: 10/238,064

(22) Filed: Sep. 10, 2002

Related U.S. Application Data
(63) Continuation-in-part of application No. 09/504,805, filed on Feb. 16, 2000.
(63) Continuation-in-part of application No. 09/711,682, filed on Nov. 9, 2000, now abandoned.

(51) Int. Cl. A61K 31/70; A61K 31/7076

(52) U.S. Cl. 514/23; 514/46

(57) ABSTRACT

This disclosure is for the nutrient, D-ribose, as well as other nutrient precursors of ATP and NAD when desired, to be administered with one or more anti-microbial agents for the purpose of enabling the anti-microbial agents to function better in combating infection than if the D-ribose were not given, plus enabling the nutrient D-ribose to improve the ability of the cloaking protein Sir2p to protect genes from attack further strengthening the immune system against infection.

### RELATED APPLICATION

This patent application is a continuation in part of patent application Ser. No. 09/504,805, “The Use of D-Ribose to Improve Cellular Hypoxia and to Better Absorb Medicaments and Nutriceuticals” and Ser. No. 09/711,682, “Using de novo D-ribose to spare NAD in the synthesis of ATP”, which applications are abandoned.

### FIELD OF THE INVENTION

This invention is in the field of increasing the resistance of humans and other animals to infection and aging, both by a direct anti-infective action and by better promoting the inherent capability of genes to protect themselves.

### BACKGROUND OF THE INVENTION

After Sir Alexander Fleming initiated the modern era of antibiotic therapy by discovering penicillin, a sensation was created by penicillin's effectiveness during World War II and as a result, a myriad of similar products ended many epidemic diseases like tuberculosis in the United States following the war. Although penicillin is based on the antibiotic molecule from the species Penicillium puberulum and P. cyclopium called penicillic acid, the acid is toxic to animals, and a derivative of it, produced both from other Penicillium species and synthetically, penicillin, proved to have far less toxicity, although effective only on gram positive bacteria unlike penicillic acid which was effective on both. Patents were applied for the more effective of these derivatives, and the two factors needed to make penicillin a household name, effectiveness and patentability, were at hand. The reason these compounds, even though derived from natural sources were patentable and, therefore, made more marketable, was that penicillic acid, although a part of penicillin, was different in kind, with different properties. Patents could not be and were not denied on the basis that since penicllic acid had antibiotic properties and was incorporated as a radical in the molecular structure of penicillin, that derivatives of penicillin could not be patented. This fact enabled inventors of dozens of naturally made substances to hold patents.

While agents like penicillin brought forth a new kind of medical treatment, many such anti-microbial agents were toxic, including streptomycin causing nerve deafness. Some also became susceptible to organism resistance, largely because they were either overused or not used for a sufficient time to destroy more pathogens during their initial or early administrations, thus giving such microscopic organisms more opportunity to become resistant. This is a continuing problem, and the latest information concerning streptococcus resistance to erythromycin shows an alarming increase.

It is, therefore, considered advantageous if means are developed to enable antimicrobial agents to work better. Such means are ordinarily called potentiation, the ability of a combination of substances to work better for a specific purpose than any one separately. In some cases, using more than one antibiotic at a time enables a better result to occur than if only one were given. The treatment of tuberculosis employed this concept and in HIV infections more than one antiviral agent are frequently used together. Many antibiotic ointments contain as many as three antibiotics used together. This kind of potentiation usually refers to two drugs such as antibiotics, that when combined, are more effective than when each one is used separately, but each drug has the same purpose.

Another definition of potentiation would be required if a different kind of drug, not an antibiotic in itself so not capable of direct anti-microbial action by itself, enabled an antibiotic to provide higher plasma levels of the antibiotic or in some other non-antibiotic way increased the effectiveness of the antibiotic or antibiotics. For example, penicillin, is given with probenecid, because probenecid delays the excretion of penicillin so renders higher plasma levels and, therefore, tissue levels of the antibiotic. By this property some drugs can reduce the toxicity of an anti-microbial agent when given so enable the anti-microbial to work better by being in the system over a longer period of time or in higher plasma levels over a shorter period of time or both.

Then there is a third type of potentiation with respect to combating pathogenic microbes that would use both of the above definitions. It would be anti-microbial by promoting the production of the body's main internal anti-microbial weapon. This tactic is the direct destruction of the pathogen by oxidation of its vital components with superoxide made by cells of the immune system, the principal one affecting this endeavor being the neutrophil. In this case, the potentiating substance would have to be a precursor to the production of superoxide, the body's own most important antibiotic or anti-microbial agent, enabling the greater production of superoxide in neutrophils and other leukocytes. This third kind of potentiation would apply when it is difficult for the body to make the required molecule or to make an essential part of it. The name for these kinds of substances can be confusing. If they do not have a phosphorous atom, they are nucleosides and if they do, they are nucleotides. In this application, the adenine nucleotides, cyclic adenosine monophosphate or AMP as hormone messenger, adenosine tri phosphate or ATP as the energy molecule, and coenzyme phosphorylated nicotinamide adenine dinucleotide or NADPH for superoxide, are the molecules with the greatest ability to fight infection either by themselves or by potentiation so to aid pharmaceutical anti-microbial agents. Ribonucleosides like coenzyme NAD have indirect potentiating uses by protecting genes and are included in this application as such. Conversely, adenosine, a neutral synthetic molecule and a product of hydrolysis, so is a drug, not a nutrient, is not anti-microbial, does not play an entry role in the Dickens shunt, directly making ATP or NADPH. Although ribose is part of it, it is not a natural ATP precursor.

For these special natural nucleotides to gain the initiative over microscopic pathogens by being made faster, requires that this key precursor, ribose, that the body synthesizes only with great difficulty, be supplied de novo. Care must be taken not to confuse ribose with its ribonucleotides. Like penicillic acid and penicillin, one of which is incorporated into the other, but each have different properties, ribose has separate properties from all its nucleosides and nucleotides, although it is incorporated in them. Therefore, this most difficultly made precursor for these important nucleotides that facilitate the formation of an anti-microbial agent in immune system cells, would fall into the first category for potentiation by making it easier for the body to produce these nucleotides and ultimately the major anti-microbial product, superoxide. The particular nucleotide, coenzyme NADPH, and its basis, nicotinamide adenine dinucleotide or NAD, are in short supply for a variety of reasons but primarily because the radical ribose in their composition is not available in food, is difficult to make, and is in great demand by the body. Thus, their anti-microbial potentiating action can be thwarted by high demand and low supply of this endogenous radical which we intend to make available as a nutrient in its neutral uncharged form so that most of the hexose monophosphate shunt or pentose phosphate pathway (Dickens shunt) for its synthesis can be bypassed.

In addition, there is this other action for this difficultly synthesized molecule that might fit more into the second category. This different nucleotide that is made up in part by this difficult-to-make component of it, adenosine triphosphate or ATP, provides for the ability of the immune system to have sufficient energy at time of need because of infection, to make the anti-microbial agent itself and also to make antibodies for later on in the infection. Since this nucleotide supplies energy for all cellular purposes, including for the immune system, its precursor would satisfy the means for the second definition of potentiation by making it easier for any anti-microbial agent including drugs to fight the infection because of increased immune system energy needed to make the first nucleotide. Contrast the fact that when adenine and ribose are combined as a nucleoside, it has entirely different actions in the body than as a nucleotide even though they both have one ribose and one adenine incorporated in them. Synthetic adenosine, must be given I.V. and is toxic in doses that precursors ribose and adenine are not. Obviously, ribose as a radical is different from ribose as a free molecule, and only the free molecule is a natural precursor.

This disclosure is to describe the overall potentiation of this precursor, the free molecule, nutrient D-ribose, which with adenine makes both nucleotides mentioned above. Synthetic nucleosides like the drug adenosine do not exist separately as free nucleosides in vivo. Ribose and adenine individually, not first combined as adenosine, are required to make superoxide, but ribose is not available directly from food. To make ribose available faster in the body so that superoxide is available faster to combine with the pharmaceutical anti-microbial drugs designed to destroy pathogens or render them harmless, is one reason for this disclosure.

We will describe means to enable such a combination of one or more pharmaceutical anti-microbial agents to be used together with this natural substance, classified by the FDA as a nutrient, although it does not exist free in nature's plant life but only as a radical and has to be synthesized in vitro from glucose by use of a recombinant DNA process or from other readily available nutrients that contain easier-to-separate ribose in their molecular structure such as riboflavin. Whatever way it is derived in vitro, in this disclosure we intend to administer de novo D-ribose as a nutrient and only in its free uncharged form of a pentose, a five-carbon atom sugar. Once inside the body it is free to combine with other molecules as a derived radical, but this disclosure is concerned about the fact that it is administered whole, without being a specific radical at the time of its administration in vivo.

In the body, it forms part of two different radicals with respect to nucleic acids, DNA (deoxyribo nucleic acid) and RNA (ribo nucleic acid). Besides DNA and RNA, it becomes a part of other molecules to form nucleosides and nucleotides in the body, its universal nature showing up also in the vitamin riboflavin. The nucleotides in question are called ribonucleotides, but it is time-consuming for the body to make ribose, so sometimes it is in short supply, usually when needed most

The body's most important metabolic nucleotide is ATP, providing cellular energy from glucose through high-energy phosphate bonds, being synthesized first by having a carbon atom from glucose removed through the Dickens shunt. Since ribose is able to form a key part of many structures that involve the immune system, it can form those nucleotides that are classified as immune system response-enhancing agents. Ribose is converted to a ribofuranosyl in combination with a purine such as guanine or thymine, which have been described as immune response-enhancing agents. Combining with phosphorous, ribose is converted to ATP by combining with the purine, adenine, to form phosphorylated adenosine. By providing energy to the immune system under attack, ATP becomes an immune-response enhancing agent because of the extra energy needed when it is under attack, which includes up to 50 times more respiratory oxygen than when leukocytes are resting. The principle cell performing this task is the neutrophil, which needs both ATP for the energy and leukocyte NADPH (reduced nicotinamide adenine dinucleotide phosphate) oxidase for enzymatic action to convert oxygen to superoxide.

Finally, ribosyl derivatives have been used as anti-viral agents. Inosine, a ribosylhypoxthanine nucleotide, is one of these that was combined with a complex benzoate derivative, pranobex, and used as an antiviral agent. Thymine ribofuranosyls were used successfully for virally caused immunodeficiency syndromes. In this case the nucleotide actually inhibits the reverse transcriptase conversion of the deoxyribo nucleotide (DNA) to the ribo nucleotide (RNA) so the RNA virus could replicate at the expense of the targeted cell (in this case most often the T lymphocyte). While it might be argued that this is an antiviral action alone, some of those skilled in the art have maintained that it can be both directly antiviral and immune response-enhancing in addition. This interpretation appears to be confirmed in Goodman's U.S. Pat. No. 4,746,651, “Anti-microbial Chemotherapeutic Potentiation Using Substituted Nucleotide Derivatives”, even though he used other purines than thymine. These disclosures, however, do not use the administration of ribose as a whole neutral molecule, but only as a radical in the derivatives of such administered nucleotides which themselves become the whole neutral molecules when administered, with ribose only a part of the larger molecule. Just like the basic molecular structure of penicillic acid is incorporated into that of penicillin, a much more complicated molecule with different properties, so too is ribose incorporated into ribonucleotides, for much more complicated molecules with different properties.

At present, the principal therapeutic use for whole, non-radicalized molecular de novo D-ribose is to reduce the ischemia of heart muscle by increasing the rate of oxidative phosphorylation so that the recovery time from lack of perfusion for such muscles is shortened. The same is true of skeletal muscles following exercise, but it is not life preserving here. In the case of the myocardium, ADP and AMP levels rise transiently during ischemia but decrease as they are dephosphorylated into metabolites (adenine, inosine and hypoxanthine) that easily diffuse through the cell membrane and are washed out of the myocardium during reperfusion. Since these metabolites become no longer available for the salvageable synthetic pathway for the re-synthesis of ATP, the de novo synthesis of ATP from glucose with its heavy dependence on the coenzyme system of NAD and its derivations is re-instituted. This process is slow getting to PRPP (phosphoribosylpyrophosphate), and de novo D-ribose is administered as a whole, neutral molecule to shorten the time considerably. St. Cyr, et al in U.S. Pat. No. 6,218, 366 B1, covered the use of ribose to raise the threshold of hypoxia but failed to disclose that ribose is also valuable to make NAD, the use of which this disclosure seeks to provide. They also failed to suggest that because I.V. synthetic adenosine dilates non-occluded coronary arteries, it gives credence to the fact that by ribose enhancing perfused ATP, it may also slowly help increase coronary circulation in the hibernating segments of the heart, even by an intrinsic vasodilating action also.

This invention is designed to overcome the deficiencies of previous applications and inventions by employing a way to make anti-microbial agents work better by supplying nutrient means to produce more cellular energy and increase superoxide production in the immune system under attack, while at the same time providing the means to control the production of superoxide where it is not desired or needed outside the immune system.

### BRIEF SUMMARY OF THE INVENTION

A group of anti-microbial agents available in 1985 are disclosed in U.S. Pat. No. 4,746,651 mentioned above. This patent first disclosed that anti-microbial nucleotides, of which both glucose and ribose were amongst its radicals, could work with other kinds of antibiotics to make the combination work better than either alone. Newer kinds of these agents include the reverse transcriptase nucleotide inhibitors for HIV, the most successful of which employ ribose as a radical. Ribose does not act independently in a nucleotide but rather as the nucleotide as a whole does, but when neutral (as to charge) molecular D-ribose is administered, the body has two fundamental precursors for biochemical processes available to provide relief from infection.

The first is to provide de novo D-ribose in order to increase its availability for the salvage of the nucleotide, ATP, in order to get more energy into immune system cells of the host. By having ribose available for administration so that its radical can be used to make other nucleotides in vivo, it can render immune system cells more able to be response-enhanced by the molecular combination of ribose with the purines and niacin needed to make such nucleosides and nucleotides as NAD and its derivatives. The process occurs slowly, not from purines provided by dietary means, but from glucose making ribose. The process to make new molecules of NAD for the immune system's superoxide is very slow when food is the only precursor for ribose via the Dickens shunt, with ATP being in great universal demand, and it needs NAD coenzymes.

The second is by providing more of the actual nutrient precursors by themselves at the optimum time to increase NADP, which is the nucleotide that makes possible, leukocyte NADPH oxidase. These amino acids can be provided separately to provide the nitrogen-containing precursors in order to make the purine part of ATP and its derivatives, as well as for the same role in NAD and its derivatives. Amino acids also provide for the protein cytochromes needed for electron and oxygen transfer. Of course, these amino acid precursors and phosphorous are readily available from food in general as are metabolite precursors of adenosine such as inosine and hypoxanthine, but not ribose. Unfortunately, free ribose is not salvaged from substances like inosine or DNA for that matter, so is not quickly provided from food, requiring much time and effort to be synthesized from glucose or glycogen or even from fat and protein breakdown. Nevertheless, in case of infection, supplying these amino acid precursors as well as de novo D-ribose will optimize performance by the immune system, even if providing de novo D-ribose does an acceptable job by itself. Supplementation of amino acids through protein is especially valuable if little food is being used by the targeted host because of lack of appetite. Since under certain circumstances ribose has an appetite-suppressing action, it must be administered properly for the indication being treated.

What is important is that for anti-microbials to work best, they need the help of the immune system, and the immune system employs superoxide species as a first resort. While the immune system will synthesize antibodies from amino acids, these take time to provide, but superoxide is readily made and relatively quickly, as long as the required nutrient precursors for the enzymes are present and devoted to making more NAD relative to ATP. This ideal situation occurs best when the host is asleep and requires minimal muscular activity to trigger ATP synthesis.

This brings us to the role that the respiratory ribose-containing enzyme, the nucleoside NAD plays along with its nucleoside and nucleotide derivatives. In the exchange of electrons and phosphorous, NAD+, NADH, NADP+ or NADPH facilitate an anti-microbial or anti-infective action as well as a protective action. This is especially significant with leukocyte NADPH oxidase, but the entire enzyme system, including the protein dehydrogenase enzymes, is vital, yet half of the coenzyme NAD molecule, the “D” or dinucleotide part, is difficult to synthesize and its precursor not available in food as the dehydrogenase enzymes and the “NA” are.

As was disclosed in patent applications Ser. No. 09/504,805 and Ser. No. 09/711,682, now abandoned, the hexose monophosphate shunt otherwise known as the pentose phosphate pathway uses NAD extensively as part of the enzymatic procedure to take glucose to AMP and then ATP. Also some protein synthesis requires NAD for enzymatic processes including those involving DNA. NAD in its common oxidized forms NAD+ and NADP+ and its respective reduced forms NADH and NADPH are essential to exchange electrons with hydrogen in many enzymatic reactions, including manufacture of cloaking proteins for genes, and with phosphorous atoms in energy conversion and in making superoxide in leukocytes.

NAD is essential for the kind of protein synthesis involving silencing genes as reported in the Sep. 22, 2000 issue of “Science Magazine” by investigators at Massachusetts Institute of Technology, pages 2126 to 2128. When the silencing gene SIR2 encodes the protein wrap for genes Sir2p, requiring NAD in the process, NAD is less available for this synthesis if glucose is available to synthesize ATP and de novo ribose is not in order to minimize the need for the shunt. Apparently, this protein wrap of genes is necessary for their silencing and not to have it at an optimum level causes unwanted access to the genes and resultant aging. Immune system cells are also targeted. It is, thereby, believed that the increased longevity induced by calorie restriction is due to more activation of Sir2p by NAD when less activation of ATP by NAD happens, also keeping the immune system stronger.

When glucose is available in the cell, without enough de novo D-ribose being present at the same time as the activation process is going on, the cell will opt to produce ATP through the hexose monophosphate shunt instead of Sir2p. Now even though NAD, or niacin coupled to adenine, coupled to two riboses, is what is used for Sir2p synthesis (as well as one adenine and one ribose for ATP) ribose is in high demand in all cases, is hard to synthesize, and not available quickly from food. There is no reason not to believe that the same dynamics are present with respect to making the leukocyte NADPH oxidase. NAD can be in short supply both with respect to protecting genes and synthesizing the leukocyte NADPH oxidase when the demand for ATP is excessive as it is with entry infections in the potentially sick, and hunger in the healthy. This disclosure seeks to provide a way that both enough ATP can be available while NAD can be increased also, by administering de novo D-ribose. This will not only retard the rate of aging in the healthy, but enable leukocytes to become more effective phagocytes with acute entry infections by producing more superoxide species, faster, early in the disease.

On the other hand, this is a two-way street. We want superoxide in the neutrophils but not in fatty layers of the mitochrondria via leaked electrons. Having the means to better cloak and silence genes by more availability of NAD and its derivatives, at a time when superoxide is both a weapon to fight pathogens and a dangerous free radical to age genes is of double benefit if superoxide can be limited in locations it ordinarily does not belong and becomes harmful as a result. By having plenty of de novo D-ribose available, extra leaked electrons from the respiratory electron transfer chain going to react with molecular oxygen to make superoxide can be minimized in organ and muscle cells, while more superoxide can be made inside leukocytes. If the body is intent on producing tediously more ATP than is necessary at the expense of the health of genes, it is also likely to produce more ATP tediously at the expense of the leukocyte NADHP oxidase at the further expense of the optimum ability to combat infection.

Therefore, with de novo D-ribose, ATP production is shortened with less superoxide leaked in the wrong places, and more NAD can be synthesized more rapidly because the ribose is already there. In effect, de novo-D-ribose becomes an indirect anti-microbial agent all by itself if it can enable more superoxide to be employed directly by the leukocytes and at the same time allowing for fewer leaked electrons via the Dickens shunt so less superoxide to destroy genes and more cloaking protein, Sir2p, made to fight superoxide already there. Ribose provides faster metabolic energy from faster-made ATP and more leukocyte superoxide faster from faster NADPH availability.

Thus, when de novo D-ribose is ingested it is rapidly taken up by cells and phosphorylated to ribose-5-phosphate with the aid of NAD coenzymes. This eliminates the need for considerable NAD used in the hexose monophosphate shunt, which can then be used to both make NADPH superoxide in leukocytes and afford more NAD protection of cells from superoxide and other free radicals by activating the Sir2p process to protect and decrease aging of the genes and reduce their susceptibility to being damaged by infective agents.

This brings up the paradoxical role that ribose plays with respect to appetite. Ribose is offered to consumers primarily because it is rate-limiting in the production of phosphoribosylpyrophosphate (PRPP), a precursor for salvage and for de novo adenine nucloeotide synthetic pathways, which maintain, AMP and ADP (adenosine diphosphate) for re-synthesis of ATP. The high-energy bonds of ATP are the direct source for muscle contraction including myocardial contractions but also energize every cell in the body, especially the immune system under attack. The presence of de novo D-ribose not only enables ATP to be synthesized much faster, but its presence also serves to stimulate the salvage of these nucleotides. With this in mind, a curious thing has been observed. If D-ribose in an aqueous solution is sprayed forcibly by pump action on the tongue during a meal, it causes the appetite to abate in many people, especially those who are overeating. However, if the ribose is swallowed, there is far less such effect. The only explanation for this is that the salvage of these nucleotides especially ATP is stimulated by forcibly spraying the ribose onto the tongue. This causes the stomach to send out less of the hormone ghrelin and a full feeling ensues with less food. If a person is ill, care must be taken not to spray ribose forcibly on the tongue by pump action or compressed gas, but rather have it be swallowed immediately or given parenterally.

Let us return to leukocyte NADPH oxidase. This complicated membrane-associated enzyme that catalyzes the reduction of oxygen to superoxide in the immune system, employs the reaction 2 O₂+NADPH→2 O₂⁻+NADP⁺+H⁺ to make superoxide species available for white blood cells when the host is under attack by pathogens. The leukocyte NADPH oxidase's load of superoxide is delivered onto internalized microorganisms and into the extracellular environment. It cannot be overemphasized that whereas, providing NAD to make more Sir2p available will prolong life over a long period, providing NADP to have more leukocyte NADPH oxidase available can help spare life at the time of an acute effort to end it by pathogens. Thus, while we are more concerned with making NAD available for leukocyte action by making more of the rapidly available superoxide for them to use, this disclosure also provides faster NAD for non-immune system genes by shortening the overall pathway to Sir2p. This helps the body fight infection in general by having more antioxidant resources available to fight harmful free radicals in general by eliminating at the start some harmful oxidants formed during the pathway. When the host is under attack by microbes, every benefit for the host counts.

Although NADH as a nutriceutical is supplied over the counter, it has had a mixed success. In many cases supplying a final nutriceutical product to the body in small amounts because of expense and toxicity is not as effective as supplying the actual nutrient precursors in relatively large amounts because nutrients are better absorbed. Sometimes even a relatively simple molecule like glutathione is not nearly as absorbable into cells as are its amino acid precursors. Nor do more complex molecules always pass the blood-brain barrier effectively. Nutrient precursors have much fewer such problems, the major problem being not having enough of them available when needed, especially when one nutrient is difficult to make yet is in great demand. This is especially the case when the body and its immune system are under attack by pathogens. The capability of having enough ribose nucleotides cannot be overestimated in general but when the host is under attack by microscopic pathogens, having enough becomes life saving. This is because ribose nucleotides play a such prominent role in DNA structure and also in cellular energy metabolism especially ATP synthesis but also in immune system response-enhancing agents of many varieties ranging from hormone messengers such as cyclic AMP to coenzymes such as NAD.

The role of NAD in protecting all cells including those of the immune system from attack by oxidants is just now being given its due consideration. Limiting the diet so less glucose becomes available dietetically, enables more NAD to be provided to the genes of healthy animals that consume less, but still enough, food. It also enables less unnecessary ATP to be made so that NAD can be made or diverted. These mice were forced to eat less by food not being available. When plenty of food is available, mice are urged to eat as much as possible by internal mechanisms involving feast and famine, just like human beings where the hormone is called ghrelin. Ghrelin is an endogenous growth hormone centered in the stomach and urges humans to eat so that there will be plenty of ATP, even if the body does not need extra ATP. This behavior is important to keep in mind also when one has an acute infection, because lack of appetite can affect some people, and care must be taken not to stimulate appetite loss in these people. Therefore, since it has been reported over and over, that when two groups of laboratory mice are fed the same nutritious diet, only with a decreased number of calories in one group, the reduced-calorie group lived considerably longer than the other and stayed in better health, obviously the reduced-calorie group had stronger immune systems but without having infections to complicate the issue. Nevertheless, in order to help the person who is infected we must consider what happens to healthy people with respect to diet and the availability of nucleotides.

Thus, the hexose monophosphate (Dickens) shunt otherwise known as the pentose phosphate pathway uses NAD extensively as part of the enzymatic procedure to take glucose to AMP, then ATP then cyclic AMP and also NAD to provide NAD+ and NADP+ and respective reduced forms of NADH and NADPH. Because as stated above, NAD in its common oxidized and reduced forms is essential to exchange electrons and hydrogen and phosphorous atoms in energy conversion, it takes precedence over the kind of protein synthesis involving silencing genes with the immune system genes also obviously being deprived of such protection. When such electrons are leaked from mitochondrial pools and pass into fatty layers of the cells, they unite with oxygen to form the oxidant and free radical, superoxide, this time in the wrong place.

Only with respect to the skin has ribose been used for healing purposes, and these are for wound healing in which ribose in combination with other nutrients improved the rate of healing. We have found that ribose by itself will improve the rate of healing of clean wounds, and when combined with antibiotics we have shown that it will improve the rate of healing infected wounds. It then became reasonable to assume that this combination should be extended to internal wounds.

Whenever a successful infection of an animal host ensues, microorganisms such as viruses, rickettsiae, fungi, parasites and bacteria multiply and in so doing can cause serious injury to the host. Infection by pathogenic microbes initiates a number of responses in a host animal including a mammal and man. Most of the time, the pathogen is recognized as foreign by the host mammal's immune system thereby activating either the humoral or cellular response or both of the host. As a consequence, antibody-producing immune system cells are stimulated to produce and secrete antibodies to combat the infecting organism. This takes more ATP than when the cells are quiescence. The complementary system that is also activated to combat the infecting organism by cells such as macrophages and neutrophils also require large amounts of energy to produce the extra superoxide.

In most cases the host is able to fight off the infection without any help and slowly makes antibodies to effect a cure, but in enough instances to pose alarm, because of insufficient cellular energy, the host's disease-fighting immune system cannot respond with enough of the killer weapon, superoxide, for the pathogenic microbes to be diminished in time to prevent host death. Other times the immune system eliminates the acute part of the infection but is unable to eliminate the pathogen sufficiently, and a state of chronic infection ensues. Both of these latter-type situations have poor results mostly because of lack of enough superoxide early on, when it counts the most. If the immune system had help soon enough, neither death nor chronic infection would be the result in many hosts, but instead there would be a cure as in the first case.

If we make the availability of enough superoxide early, more help can be provided by the use of anti-microbial agents of various sorts, and it is being proposed here to provide the whole neutral molecule of D-ribose with them and also when desired, as many as are needed of the rest of the major precursors for immune system nucleotides such as leukocyte NADPH oxidase. This, of course, means ATP for more cellular energy and NAD for enzymatic action both for more energy and for leukocyte-producing superoxide.

Although administering D-ribose alone will provide more cellular energy for all cells in need when the immune system is under attack, the immune system will receive a life-protecting benefit from the faster cellular energy de novo D-ribose provides by facilitating the synthesis and salvage of ATP at the time when pathogens are attacking. Special benefit will occur when it is administered early in the disease even before antibiotics are provided and especially during and afterwards. In addition, by providing all of the nutrient precursors for ATP and for NAD, even more protection for the body is achieved by early use.

### ADVANTAGES OF THIS INVENTION

The present invention applies certain benefits and advantages. One benefit is that a similar treatment response in overcoming a disease being treated can be accomplished by using less of the anti-microbial agent. This will enable toxic drugs to be used in lower amounts. In case a stronger response is needed, the regular dose of antibiotic can still be given along with the D-ribose and, if desired, other precursors for ATP and NAD.

A further advantage of this invention is that the immune system under attack can be improved by the nutrient part of the disclosure alone. As precursors for the energy molecule ATP as well as being a precursor for natural nucleotide molecules that enable the immune system to provide an enhanced response, including NAD and its derivatives, the derived ribose radical and the others needed for these molecules can be provided by the body's cells much more rapidly. The synthesis of ATP can occur in as little as 8 hours with de novo D-ribose, rather than the long time of 72 to 96 hours ordinarily required for glucose to have removed the extra carbon atom, and the faster salvage of ATP that ribose affords, comes even quicker. Also the synthesis of NAD can be speeded by having more ribose available more rapidly.

Still another advantage of this invention is that the D-ribose alone or with its associate nutrient precursors can be given at the same time in the same delivery combination as the anti-microbial agent or in separate delivery entities and even at separate times, and such doses can be peroral or parenteral (amino acids are available for parenteral administration also), so tissue levels of each can be optimized with respect to each other.

Still further benefits and advantages will be apparent to those skilled in the art from the detailed description that follows.

### DETAILED DESCRIPTION OF THE INVENTION

The present invention contemplates one or more anti-microbial agents being potentiated by the concomitant administration of whole nutrient molecules that are not radicals of another molecule when administered and a method of their use. D-ribose is one of these nutrients, uncharged and not radicalized when administered, which can be used alone or with other nutrient precursors. The other major nutrient precursors include amino acids usually via proteins, niacin and phosphates. Other support minerals such as magnesium and multivitamins can also be included. The anti-microbial agents are one or more of any in vivo-effective amounts of FDA-approved substances designed to aid the body to reduce or destroy pathogens afflicting it. The pathogens include, viruses, fungi, gram-negative bacteria, gram-positive bacteria, acid-fast bacteria, parasitic groups, rickettsia and any other agent for which anti-microbial substances are indicated and provided. With respect to the various methods of delivery, the antibiotics can be given perorally in liquid or solid form and the D-ribose, along with the other nutrient precursors when desired, can be included within the same vehicle, or given separately. The same is true with parenteral administration; the D-ribose can be given as a separate injection or infusion, and the anti-microbial agent or agents can either be in each composition or both administered separately. Niacin and phosphates can be given in the same or different compositions, but parenteral amino acids are best given by intravenous infusion. Whether to do one or the other will depend on the factors present. These can include incompatibility of substances combined in the same delivery composition and intolerance by the patient to combined delivery in the same composition. Finally D-ribose can be administered on the tongue forcibly by pump action, usually by compressing air through a long tube in an air-tight container holding D-ribose dissolved in water, which containers commercially are readily available in various sizes as ordinary spray bottles. Care must be taken that this route not be used in anorexic patients, although it can be used to help prevent overeating in the healthy in order to encourage Sir2p synthesis by reducing ATP production from unneeded excess food.

The preferred and most essential embodiment of the nutrient part of the invention that is needed to enable the most-timely production of ATP and NAD because it is the most time-consuming for the body to synthesize, is the pentose D-ribose as a neutral whole molecule. The term “D-ribose” is therefore intended to include such similar molecules that are readily converted to D-ribose, hence are immediate precursors of D-ribose. Xylitol and ribulose are included amongst these pentose precursors. For maximum effectiveness other nutrients, e.g. amino acids, niacin and phosphates, may be supplied in vitro also, although without the de novo D-ribose they would be ineffective to potentiate the anti-microbial agents in the timely manner that D-ribose can, because they do not need time-consuming synthesis. Nevertheless, de novo D-ribose may become even more effective when these associated nutrients are administered at or near the same time.

Exemplary anti-microbial agents and their dosage schedules are listed in the annually published “Physicians' Desk Reference” and its updated addendums.

D-ribose is an FDA approved over the counter nutrient in doses of 20 grams or less a day. Physicians may recommend larger doses when required. Whereas, D-ribose is provided over the counter in the form of a powder or granulated, it can be dissolved in water and used that way or made into an intravenous solution just like 5% dextrose and water. It also can be given by inter-muscular injection. As much as 10% D-ribose in sterile water can be used parenterally with tolerance by the veins. This much needs only be given when acutely ill people are being treated, and the anti-microbial agents are being given parenterally during the same time period. This includes infusion of either ribose, anti-microbial agents or both into the peritoneum and abdominal cavities when indicated, but nutrients and anti-microbial agents should not be infused into the abdominal cavity without further research with each substance to prove safety and effectiveness.

While superoxide is essential with respect to leukocytes targeting pathogens, superoxide can also target vital life structures including the genes. Therefore, when the silencing gene SIR2 encodes protective protein wrap Sir2p for genes, requiring NAD in the process, NAD is less available for this synthesis if glucose producing ribose is being used to synthesize more ATP than needed at the expense of NAD enabling the synthesis of Sir2p for gene protection including those in the immune system. When de novo D-ribose is given, not only will ATP be synthesized and salvaged faster and more so, but the same benefit will accrue to other nucleotides having a ribose radical such as cyclic AMP and NAD. Therefore, when a host is fighting an infection, all nutrient precursors for NAD need to be more available than when the host is well. When minimal glucose is consumed, more NAD becomes available. During sleep there is minimal demand for glucose to make ATP for skeletal muscles, so this process works best while the body is detoxifying during sleep. Thus, although it needs to be administered frequently when awake, de novo D-ribose also needs to be made available either alone or with the other nutrient precursors of NAD just before sleep, optimally with little or no competing carbohydrate ingested for several hours prior to sleep, so less ribose need be synthesized and wasted by the cells for energy through the hexose monophosphate shunt at the expense of NAD production. The immune system under attack needs both more NAD and ATP as fast as it can get them, and ribose enhances the availability of all such nucleosides and nucleotides, both as to amount received and the speed of availability. The faster this energy is supplied, and the enzymes made, the better and more effective the healing.

D-ribose is the only precursor for NAD and ATP that is made slowly from carbohydrates and stored glycogen, as well as from fat and protein. Therefore, only D-ribose can be used more effectively alone, because the other NAD and ATP precursors are available in food and do not need the time it takes for a carbon atom to be removed from glucose, as is the case with ribose synthesis. Nevertheless, it becomes obvious that if precursors besides D-ribose, such as amino acids, vitamin B-3 (niacin), dietary phosphorous and other key minerals, are supplied to the body in close proximity to each other, there could be even less delay in making superoxide, especially being administered after a short fast before sleeping. Ribose, amino acids, phosphorous and niacin would include three of the precursors for NAD and NADH and 4 of the precursors for NADP and NADPH. Since these all would include the one that takes so much time to synthesize, ribose, more usable precursors of all will be available to aid the synthesis of NAD and its derivatives for better immune system action and anything else NAD and its derivatives are used for.

There is one other major use for the nutrient precursors of adenine, which are mostly supplied from the ingestion of protein, and that is to make the respiration hemoproteins, called the cytochromes, which in time of infection are especially needed to enable leukocyte NADPH oxidase to work by making usable oxygen available in the process of respiration.

For less serious infections the D-ribose can be given orally in divided doses. It can be included with other nutrient precursors of ATP and NAD in the same or separate compositions. Since it is rapidly absorbed through the intestinal mucosa and is used up rapidly by the cells which constantly demand that ribose be made from glucose so will use up the ribose rapidly, it works best when given frequently so that ribose is constantly available for the cells. The other precursors can be given frequently also in the case of acute infections. Nevertheless, given less often, such as only twice a day for convenience and compliance, other nutrient precursors of ATP and NAD may be useful when a physician so dictates. For a single administration for this disclosure, given before bedtime would be the best single time. Nevertheless, with acute infections D-ribose should be given as often as 4 times a day by mouth, even if the rest of the nutrient precursors are provided less often.

If it is being provided parenterally for serious acute infections, it needs to be made continuously available for best results. For the most acute infections giving it at the rate of 180 milligrams per kilogram per hour may be needed. For less severe infections reduced amounts can be given. Once the combination of the anti-microbial agents and the ribose have improved the condition of the patient, the ribose may be reduced even to only one gram, but still a total of 20 grams per day in divided doses should be maintained for a week in most cases. Even 60 grams or more may be administered during a day when large amounts of anti-microbial agents are being administered for an acutely ill patient.

The non-nutrient anti-microbial agents are given in the manner and dosage that the PDR or the FDA recommends except when larger amounts are deemed necessary by the attending physicians. Anti-microbial agents should never be discontinued prematurely because this increases the likelihood of enabling the microbes to become resistant. Only if the patient cannot tolerate them should they be discontinued prior to the time that is ordinarily recommended for optimum usage.

Usage over time will enable us to tell how well using D-ribose with the anti-microbial agent or agents reduces or destroys the microbes, because more cellular energy is available to the cells of the immune system with more superoxide production. If an enhanced response greater than would be the case without using the combination, then shorter treatment times become possible. This would go along with the possibility of smaller amounts of anti-microbial agents being given. The entire regimen of anti-microbial agents, when given with D-ribose to enhance the availability of leukocyte NADPH oxidase making superoxide species, can be reduced in quantity because of the maximum availability of superoxide by these immune system cells by such a protocol. This will then have many dosage benefits. Since the combination would be more effective, it allows reducing the total amount of anti-microbial agents that will be beneficial by having fewer side effects from toxicity. On the other hand, if the antibiotic needs to be taken for a long time, reducing the amount of it that is needed can result in greater tolerance by the patient to the anti-microbial agent itself. A further benefit can be had by enabling an even higher level of anti-microbial agent than would be tolerated ordinarily to be used more safely in greater amounts when absolutely necessary. Another reason for this is that the ribose and the other ATP and NAD nutrient precursors also work to enhance the production of immune system cellular energy in general as well as leukocyte NADPH oxidase in particular to make superoxide. The increased energy will enable the anti-microbial agents to work faster so as not to be needed as long in the higher doses.

Let us discuss non-ribose precursors for NAD coenzymes and ATP. These include amino acids that make the protein enzymes, such as dehydrogenases, that help synthesize the several nucleotide-forming radicals that make up the energy molecule and coenzymes themselves. The purine, adenine, which with ribose makes adenosine, is essential. While adenine can be given in molecular form, it is abundantly available from protein food along with and through its associated amino acid molecules, and because of this is available for immediate use, unlike ribose, which is only available in food in the precursor form of glucose. Carniglia disclsosed in U.S. Pat. No. 4,923,851, that actually supplying the specific amino acids in themselves along with the ribose facilitates performance by horses and wound healing in mice without anti-microbial agents. Nevertheless, for healing with infection a threat, therefore used with anti-microbial agents, these nutrients, especially D-ribose, the essential one needed for success, provide more likelihood for a successful treatment by the potentiation factor

Expense must also be a consideration since anti-microbial agents often are very expensive, so potentiation is economical. Therefore, the use of a virtually complete protein powder such as soy should do well enough orally, since it has most precursor amino acids in it. Specific individual additions of amino acids may be added if desired by the clinician who prescribes the antibiotics. Therefore, a protein powder may be included in the regimen effectively, but usually administered from a separate package than the D-ribose, because fast-utilized D-ribose may need to be given more often than the protein. Since phosphorous is very abundant in food including soy protein, it is not vital that it be given in large amounts, even though its bonds supply the energy. In the order of a gram of phosphorous is consumed by an individual daily, but if an infection is involved even though most healthy people get all the phosphorous they need, dietary phosphorous in the order of one gram a day may be added to the precursor list along with magnesium, calcium, multivitamins and other minerals. Too much phosphorous should be avoided because it interferes with calcium deposition in bone.

The final precursor for making NAD, leukocyte NADPH oxidase and NAD's other derivatives in oxidized and reduced forms, NAD+, NADH, NADP+ and NADPH, is vitamin B-3 (niacin) which needs to be made more available to aid the synthesis of a maximum amount of NAD and its derivatives for better immune system action and anything else NAD and its derivatives are used for. At least the RDA value of vitamin B-3 should be used as a minimum as an additive, which is 20 milligrams. More may be given for maximum NAD synthesis, and 100 milligrams or more a day is not excessive, since 500 milligrams a day is provided for cholesterol lowering.

NADH itself from fish is commercially available with the recommended dose of 5 to 10 milligrams a day although as much as 40 milligrams per day can be used, if ordered by a physician. NADH has been reported to be used to reduce AZT toxicity. While this action is a potentiation of the effectiveness of AZT by a complete molecule, and this disclosure is about using the precursors for NAD, using NADH when available, along with its precursors may even increase the benefit. In those cases where extra expense is not a factor, the precursors for NAD as well as the basic molecule itself in its reduced form, NADH can be administered at the same time. This would tend to make even more leukocyte NADPH oxidase available in case of infection. If this makes more enzyme and enzyme precursor available than the body can use, since these are nutrients and of low toxicity, the extra unused amount that ensures maximum availability would be of benefit to ensure abundance. On a cost-effective basis providing more of the nutrient precursors is of more value than providing the molecule itself, because the precursors are better absorbed on a milligram-to-milligram basis, far less expensive.

Whereas, even if the anti-microbial agent is to be taken only once a day, the nutrient precursors of ATP and NAD may be taken more often. On the other hand, the nutrient precursors also may be taken only once a day and still get some benefit, but if that is done, it is better that they be taken at bedtime as mentioned above and no food be taken for at least 2 hours before. This way after a 2-hour fast, dietary sugar will interfere far less with nucleotide synthesis, because it will not stimulate ATP production in place of NAD production. The same is true for voluntary muscular action. During sleep, there is far less such action which targets ATP use, so more ATP will be available for the immune system, not the muscles, as well as more NAD for respiratory purposes and Sir2p synthesis.

Finally there is the role of neutrophils in infections. While the main objective of this disclosure is to augment the value of anti-microbial agents by the production of more superoxide and energy for the immune system cells, as a result of enabling more ribose and other nutrient precursors of ATP and leukocyte NADPH oxidase to be available, the neutrophils are the main cells to use the extra energy required to make extra superoxide and release most of the superoxide, having 4 times the respiratory burst capacity of other immune system cells. Upon being activated and by chemotactic mechanisms, neutrophils discover the location of pathogenic microbes, target the microbe and provide a respiratory burst that releases a variety of highly toxic oxidant species besides the main one, superoxide.

Once the infection has taken hold, these cells are depressed in number during viral attacks and increase considerably during bacterial assaults, possibly because viruses are a small target and other leukocytes become more in demand to make antibodies. Since neutrophils produce most of the superoxide, but superoxide is hard to measure as to in vivo quantity but neutrophils are easy to measure, it would appear that the unit to measure is the neutrophil, if superoxide capacity were to be estimated. If serial counts of neutrophils were done, and their counts became lower in a healthy person, it would signal the fact that more precursors for superoxide manufacture be present for the remaining neutrophils so that they would be of maximum effectiveness in the event infecting microbes should enter the host. The more anti-microbial action there is at the beginning of an infection, the better the chances are for the host to repel it. The fact that when an infection begins, the up-until-then resting neutrophils will need more glucose to be shunted through the time-consuming hexose monophosphate shunt to fuel via ATP the production of NADPH. If de novo D-ribose were already there, it would not only make ATP faster but NADPH faster. Since it is not a question of if a host is going to be exposed to an infection, but when, by doing serial neutrophil studies, people may be alerted to take D-ribose, at least once, every day, especially at bedtime. If pathogenic microorganisms should gain entrance, at least part of the delay through the hexose monophosphate shunt would be reduced significantly at a time when it could be lifesaving. Therefore, since the degree of anti-microbial capacity by superoxide could very well be related to the number of neutrophils, keeping serial track of their numbers is a simple procedure to do, and should be done.

We now can sum up a complete algorithm for the most effective series of steps available for the control of infections by pathogenic microorganisms ranging from prevention to treatment. The first step is making available the nutrient precursors of neutrophils' leukocyte superoxide oxidase as well as the superoxide made by other cells of the immune system at a lesser rate. Since only ribose is not available in quantity in food, but all other precursors for ATP and NAD are, they are readily available after absorption and can be given even in larger than usual quantities as long as the food or supplements contain them. Therefore, the first step in keeping people as free from infection as possible is to maximize the availability of the nutrient ribose precursor of leukocyte NADPH oxidase by taking it even before an infection is at hand, so the neutrophils and other leukocytes can have quick access to more of it, when stimulated by the entrance of an infective agent. The minimum amount of D-ribose taken at bedtime should be 1 gram but 5 grams is better. If the neutrophil count is low, ribose can be increased in amount just to be on the safe side, since we don't really know exactly how many neutrophils is optimum. The most optimum time to make sure de novo D-ribose is available for NAD is when making ATP is at a low level. This optimum time is during sleep, which requires that ribose be given every night in anticipation of an infection. Once an infection has gained a foothold, step-2 needs to be implemented. Treatment needs to be provided by anti-microbial agents according to the best regimen available, but the need for ribose potentiation has now increased. Now D-ribose needs to be given in substantial amounts day and night up to 60 grams and even more, along with the prescribed antibiotics. Other nutrient precursors normally available with food can be given also at any time before and infection or during it, to increase their availability for dehydrogenase production to team up with coenzymes to make as much ATP and NAD as possible or desired.

Finally with respect to the preventative part of this disclosure, during those times when an active new entry infection is not present or when anti-microbial agents are not indicated, enabling NAD and its derivatives to be synthesized faster and being more available because of the administration of de novo D-ribose by avoiding going through the hexose monophosphate shunt to achieve it, D-ribose can both help prevent an entry infection and help the silencing gene SIR2 make more cloaking protein Sir2p to protect genes and by that retard their aging and destruction. D-ribose can be given by any route, including being placed in water to be used at fitness clubs and tanning salons as well as for general distribution. Bottled water with from 2 to 8 grams or more of ribose per liter water would be a convenient way to market D-ribose or prepare it for use in doctors' offices. It will also help to reduce skin damage from

I claim:

1. The method of combining a nutrient precursor of important nucleosides and nucleotides, vital for those life processes including gene protection, metabolic energy, immune system response-enhancement and leukocyte superoxide production, when a host and his or her immune system are under attack or potential attack, with at least one non-nutrient anti-microbial agent designed to function effectively for an infection without said nutrient, but which is able to function even more effectively with said nutrient being administered also.

2. The method of claim 1 in which the pentose sugar, D-ribose, is said nutrient.

3. The method of claim 1 in which from 1 to more than 60 grams of de novo D-ribose are administered by any route within 24 hours.

4. The method of claim 1 in which said metabolic energy nucleotide is ATP and said immune system response-enhancers are NAD+, NADH, NADP+ and NADPH, all being derivatives of nucleoside NAD.

5. The method of claim 1 in which said anti-microbial agents and their dosage recommendations are listed, updated and printed annually in the “Physicians' Desk Reference”.

6. The method of claim 1 in which D-ribose and the anti-microbial agents may be given together or separately.

7. The method of claim 1 where NAD and its derivatives are both spared and synthesized to enable more Sir2p to be synthesized by the gene SIR2 to retard aging by protecting genes.

8. The method in which the oral administration of D-ribose is sprayed on the tongue to curb overeating.

9. The method of claim 1 in which parenteral administration of D-ribose is used.

10. The method of claim 1 in which oral administration of the anti-microbial agents is used.

11. The method of claim 1 in which parenteral administration of the anti-microbial agents is used.

12. The method of claim 1 in which the D-ribose is in the same delivery composition with the anti-microbial agent.

13. The method of claim 1 in which the D-ribose is in a different delivery composition from that of the anti-microbial agent.

14. The method of claim 1 in which serial neutrophil counts are used as an index of immune system superoxide production.

15. The method whereby protein powder meal is administered with D-ribose to facilitate the synthesis of NAD+, NADH, NADP+, NADPH and ATP.

16. The method of claim 15. whereby niacin is administered with D-ribose as precursors to facilitate the synthesis of NAD+, NADH, NADP+, and NADPH.

17. The method of claim 15 whereby dietary phosphorous is administered with D-ribose as precursors to facilitate the synthesis of ATP, NADP+and NADPH.

18. The method whereby D-ribose, soy protein meal, niacin and phosphorous are taken at bedtime, at least two hours after previous food has been consumed.

19. The method of claim 1 whereby the serial administration of de novo D-ribose is provided for an period of more than one day before the anti-microbial pharmaceutical agents are administered.

20. The method of claim 1 wherein said nutrient precursor is administered by itself.
